(19) United States
(12) Patent Application Publication
(10) Pub. No.: US 2014/0014122 A1
(43) Pub. Date: Jan. 16, 2014

(54) HemZem Anal Pile Driver Medical Device

(71) Applicant: John P. Callan, Lake Forest, IL (US)

(72) Inventor: John P. Callan, Lake Forest, IL (US)

(21) Appl. No.: 13/733,553

(22) Filed: Jan. 3, 2013

Related U.S. Application Data
(60) Provisional application No. 61/690,972, filed on Jul. 10, 2012.

(51) Int. Cl. A61F 5/00

(52) U.S. Cl. CPC A61F 5/0093
USPC 128/887

(57) ABSTRACT

The HemZem Anal Pile Driver Medical Device is a nonabsorbable medical minipad made from 16 ply layered facial tissue paper. The minipad is novel in that it is employed as an anal pessary using the body's own distinct anatomy, physiology, and chemistry to remain in place, and prevent the prolapse of anatomical hemorrhoids. When inserted into the anal canal, the HemZem Anal Pile Driver prevents medical complications such as itching, burning, or pain associated with, or secondary to prolapsing hemorrhoids. The HemZem Anal Pile Driver also serves as an absorbent minipad for anal mucus, and prevents soiling of clothing from such mucus, or topical ointments, creams, and gels commonly used to treat hemorrhoids. The US Food and Drug Administration classified the HemZem Anal Pile Driver as a Class I medical device on Aug. 23, 2012.

### CROSS-REFERENCE TO RELATED APPLICATIONS

This application is a continuation of U.S. provisional patent application 61/690,972 filed on Jul. 10, 2012 which is incorporated herein in its entirety. Patent application 61/690,972 was applied for by and issued to John Patrick Callan who is sole inventor of the HemZem Anal Pile Driver Medical Device.

### STATEMENT REGARDING FEDERALLY SPONSORED RESEARCH OR DEVELOPMENT

Not applicable

### REFERENCE TO SEQUENCE LISTING

Not applicable

### BACKGROUND OF THE INVENTION

Every human being has hemorrhoidal cushions—a collection of small blood vessels—micro-veins, arterioles, nerves, and dermis—at the lower end of the rectum and anus. That is normal. The hemorrhoid cushions act as a seal to create a watertight anus. As long as the cushions remain in place, an individual experiences no difficulty or symptoms. But if the hemorrhoidal cushions become unduly swollen and are displaced downward—prolapse—through the anal orifice, troubling symptoms develop. Symptoms include pain, sensation of burning, itching, bleeding, and general discomfort. Sometimes thrombosis or torsion of a prolapsed hemorrhoid—or pile as the condition is commonly called—occurs. This can lead to additional complications such as infection, perianal abscess, and anal fistula.

In addition, prolapsed hemorrhoids release naturally occurring mucus from the covering anoderm. This causes additional problems such as irritation and inflammation of bodily tissues surrounding the anus. It may also cause anal incontinence leading to the involuntary discharge or leaking of feces, and soiling of clothing. It is estimated that about 50 million Americans suffer from prolapsing hemorrhoids.

In many ways prolapsing hemorrhoids or piles are medically considered akin to varicose veins in the leg. They are uncomfortable, unsightly, sore, and worrisome—but not considered medically serious (i.e. do not decrease normal life expectancy) unless severe complications—such as heavy bleeding or varicose ulcers—crop up. Such situations are potentially lethal and require medical intervention. But average cases of hemorrhoids or varicosities do not, which is a reason why many individuals choose to simply live with their situation. Or they try to manage them by themselves with a variety of natural or over-the-counter preparations with varying degrees of success.

There are many currently available treatments for hemorrhoids, but all have drawbacks. The most definitive treatment is hemorrhoidectomy, a surgical procedure with a success rate of more than 90%. It is expensive—costs thousands of dollars—can have short and long-term complications such as infection, anal incontinence, or stricture, and is highly painful for up to several weeks after the operation. In addition, the hemorrhoids may return, negating the effect of the procedure. There are other surgical interventions such as banding, stapling, injection with sclerosing agents, or freezing. These can cause the same complications as hemorrhoidectomy. There are also nonsurgical medical and nonmedical treatments such as ointments, creams, gels, salves, and suppositories. These can provide temporary relief of symptoms but are messy, inconvenient, and do nothing to address the underlying cause of the problem—the prolapsing hemorrhoid.

This brings us to this invention the HemZem Anal Pile Driver which is designed to hold hemorrhoids in place and prevent their protrusion out through the anal orifice. When placed snugly in the anal canal the HemZem does that without a need for external attachments or fasteners. Holding hemorrhoids in place prevents the subsequent chafing of the exposed hemorrhoid or pile, and the development of swelling, and pain. The HemZem also absorbs mucus and other anal effluvia which prevents inflammation of the perianal area.

Various devices have been patented over the years with the US Patent Office to address prolapsing hemorrhoids by holding them in place. For example, see U.S. Pat. No. 5,924,423 to Majlessi, U.S. Pat. No. 4,583,542 to Boyd, and U.S. Pat. No. 5,178,627 to Hudock, U.S. Pat. No. 4,331,151 to Golden, U.S. Pat. No. 8,118,726 to McFaul Sr, U.S. Pat. No. 6,716,229 to Toth, U.S. Pat. No. 8,062,277 to Fleming, U.S. Pat. No. 6,364,852 to Lee, U.S. Pat. No. 4,263,914 to Pawlak, U.S. Pat. No. 8,211,101 to Croft, U.S. Pat. No. 4,702,235 to Gianopoulos.

These and other devices or treatment have not proved very useful or effective which is a reason to request a patent for this invention, the HemZem Anal Pile Driver Medical Device. While there are medicated anal wipes and pads on the market in the U.S. at the present time, these are designed to be used and discarded after a moment or two. They are not designed to be inserted and left in place for hours on end, and do not work as HemZem does—uniquely encoding the body's own anatomy, physiology, and chemistry—for beneficial results.

### BRIEF SUMMARY OF THE INVENTION

The HemZem Anal Pile Driver Medical Device, the invention, is a soft, rectangular, anal pessary with the approximate dimensions of three inches (7.6 cm), by one inch (2.54 cm,) by an eighth of an inch (32 cm) thick. It is inserted into the anal canal of individuals who suffer from prolapsing hemorrhoids to ameliorate their condition. In the anal canal, it is designed to conform to human anatomy where it holds human anatomical hemorrhoids in place for several hours above the dentate (ano-rectal) line of the anal canal.

The purpose of the invention is to prevent hemorrhoid protrusion or prolapse through the anal canal, and to prevent or ameliorate medical complications resulting from that, such as burning, bleeding, and pain. The invention is also designed to absorb anal mucus both as a functional component of the device remaining in place, and to block perianal irritation. In the event of fecal leaking, or emergence other effluvia from the anus, which is often associated with prolapsing hemorrhoids, the HemZem invention is designed to absorb such spillage. The HemZem invention is also designed to serve as a barrier for medical ointments, creams, and gels used in the treatment of hemorrhoids, and thereby prevent the staining of clothing from such topical applications.

### BRIEF DESCRIPTION OF THE SEVERAL VIEWS OF THE DRAWING

There are 8 drawings on 6 sheets which refer to the manufacture, final composition, and usage of the HemZem Anal Pile Driver.

FIG. 1 depicts an 8 inch by 8 inch smooth flat sheet of 2 ply facial tissue.

FIG. 2 depicts an another view of the 8 inch by 8 inch smooth sheet of facial tissue depicted in FIG. 1.

FIG. 3 depicts the former 8 inch by 8 inch smooth facial tissue of FIG. 1 that has been folded in half rendering it 4 ply with the dimensions 8 inches by 4 inches.

FIG. 4 depicts the former 8 inch by 4 inch smooth facial tissue of FIG. 3 that has been folded longitudinally in half rendering it 8 ply with the dimensions 8 inches by 2 inches.

FIG. 5 depicts the former 8 inch by 2 inch smooth facial tissue of FIG. 4 that has been folded in half rendering it 16 ply with the dimensions 8 inches by 1 inches.

FIG. 6 depicts the former 8 inch by 1 inch smooth facial tissue strip of FIG. 5 that has been cut into a 3 inch by 1 inch 16 ply tissue pad.

FIG. 7 depicts a blow up of one end of the 16 ply tissue pad depicted in FIG. 6 showing the open and closed layers of the invention.

FIG. 8 depicts the 16 ply tissue pad of FIG. 6 in the applied functional position in the human anorectal canal.

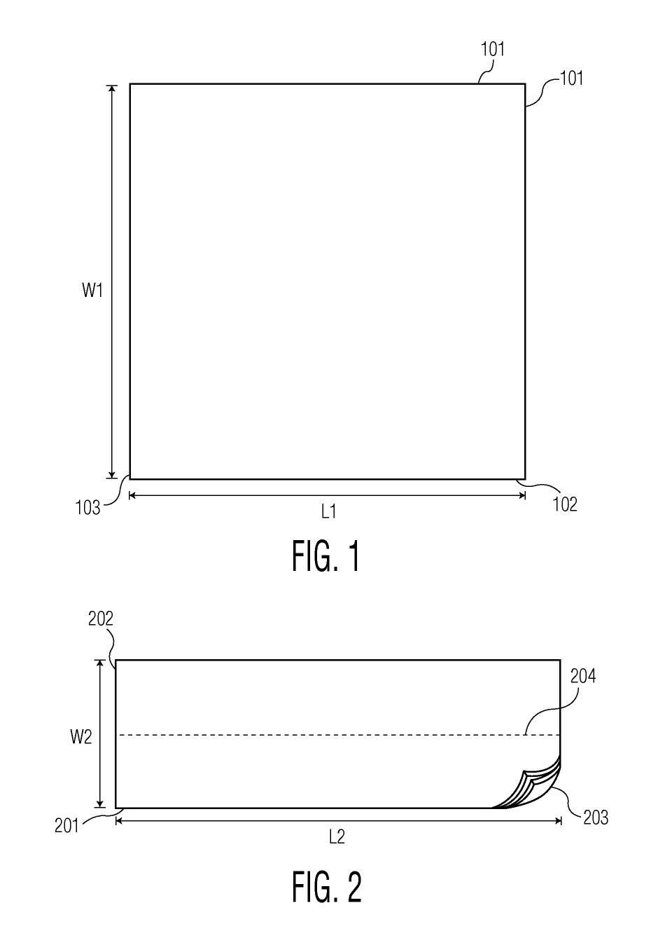

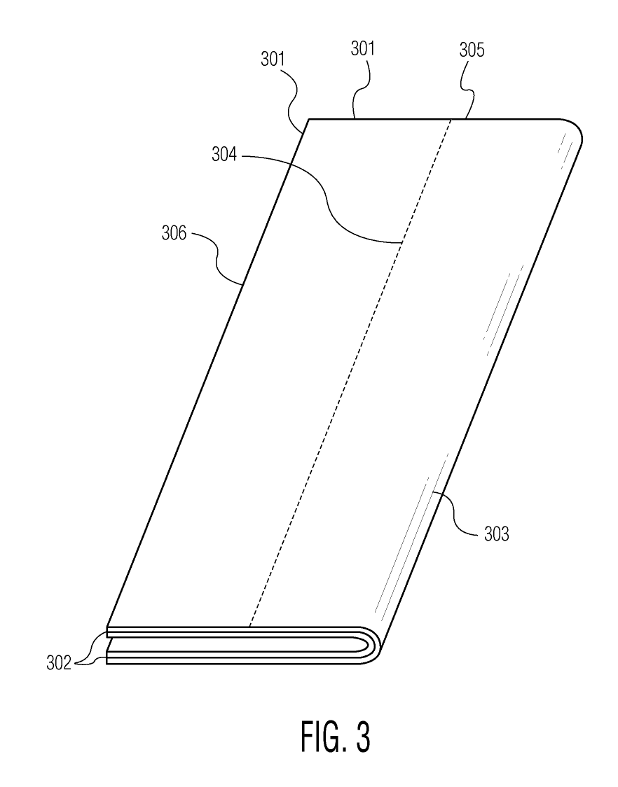

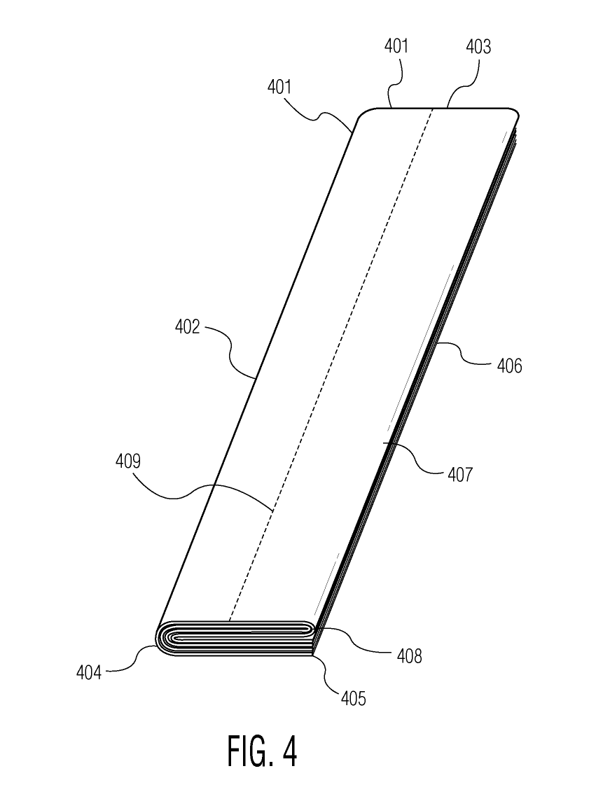

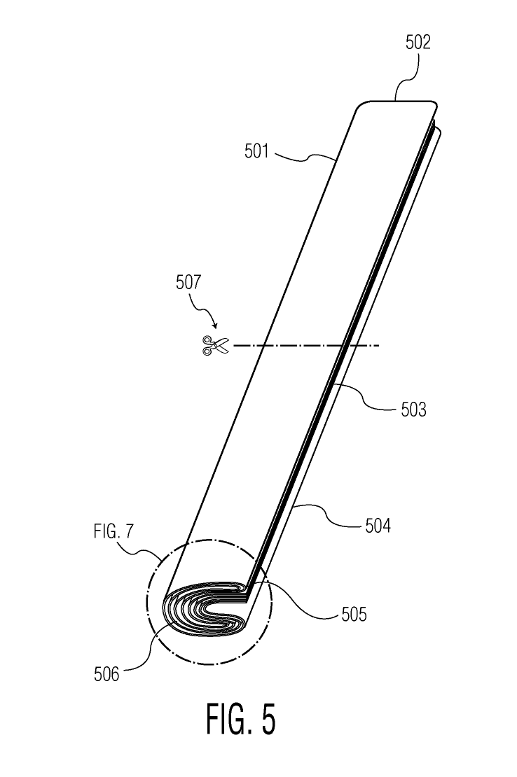

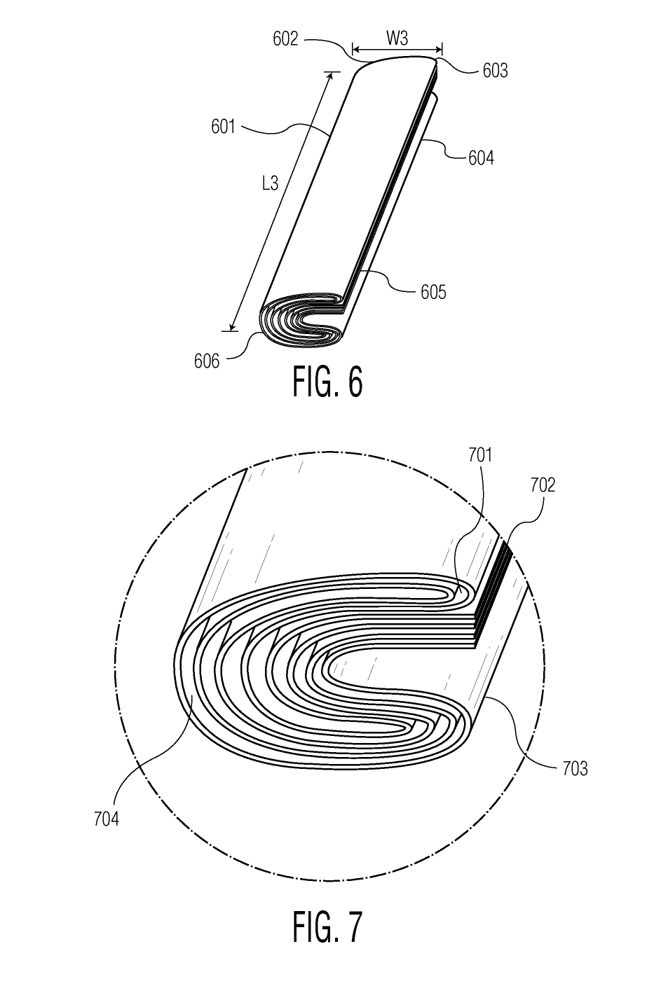

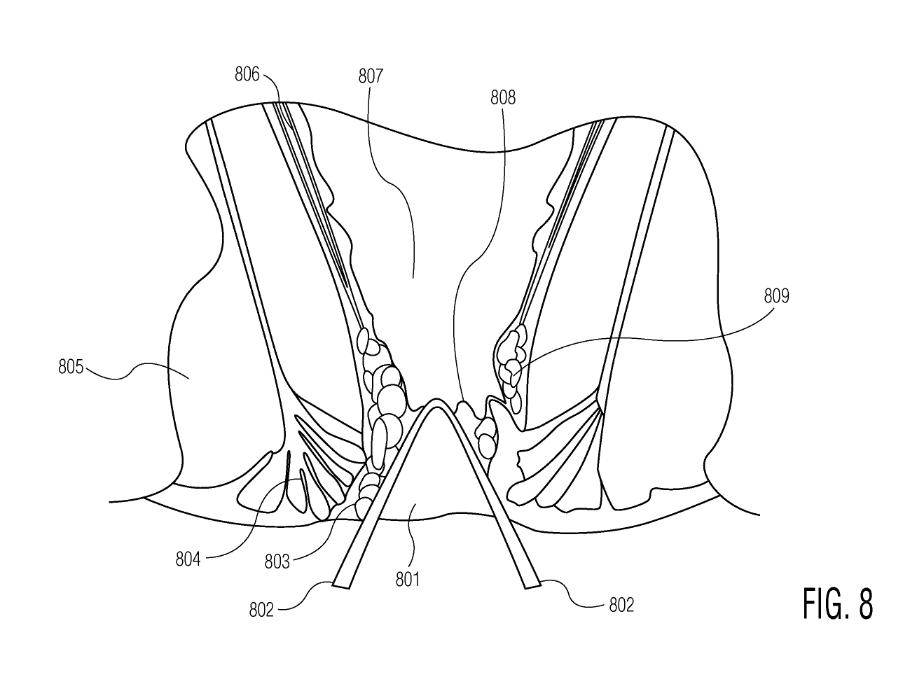

### DETAILED DESCRIPTION OF THE INVENTION

The purpose of the present invention, the HemZem Anal Pile Driver, is to support and hold prolapsing hemorrhoids in their original anatomical position without a need for additional attached supports such as special clothing, fasteners, adhesive, belts, ties, or ribbons. The invention is kept in place exclusively by the body's human anatomy, physiology, and chemistry. By holding hemorrhoids in place, the invention prevents and relieves the common complications associated with prolapsed hemorrhoids or piles. These complications include perianal irritation, inflammation, chronic itching, pain, bleeding, and infection of the anal area.

A secondary purpose of the invention is to absorb the mucus, fecal leakage, and other anal effluvia associated with symptomatic hemorrhoids. This function reduces the incidence of other complications such as compromised anal hygiene and stained clothing. The invention, a multi-ply pad made from facial tissue paper, works as an anal pessary or truss when situated in the anal canal and stays in place due to its small size, lightness, absorbency, the nature of bodily secretions, and its compact nature. The invention is held in place by the muscles of the pelvic floor, the anal sphincter muscle, human anatomy and the mucilage of the user's naturally occurring bodily mucus.

The anal sphincter muscle is unique; it is a voluntary muscle yet it is one of the few muscles in the body that remains in a constant state of contraction until it is voluntarily relaxed to permit defecation. Other voluntary muscles work in the opposite manner—constantly relaxed until flexed. The characteristic of the anal sphincter staying contracted enables the inserted HemZem to remain seated in place following insertion. The other muscles of the pelvic floor have a bowl or sling-like action and add their support to structures situated in that anatomical region including the invention when it is situated in place.

Human anatomical dissection and radiography shows that the intersection between the rectum and anal canal is not vertical. There is an angle of about 90-110 degrees between the two which is maintained by the pelvic muscles. Thus, the placement of this invention when in use is not in the vertical position but is at an angle to vertical which is an additional reason for it not being displaced due to gravity. The invention is soft and pliable and conforms to human anatomy both during exercise, and at rest which further holds it in place.

The human intestine including the rectoanal area is lined with cells that secrete mucus, a thick gluey substance, which has several roles including keeping the intestine moist and lubricated. Mucus also serves as a natural protective by keeping bacteria at bay. Mucus occurs naturally with and without the digestion and passage of ingested food. It is excreted in feces along with other bodily waste. Analysis of mucus shows it to be a mucopolysacharide and glycoprotein constructed in long polymer chains which make it a natural glue. This characteristic facilitates the action of this invention HemZem by also holding it in place. Prolapsed hemorrhoids secrete mucus causing irritation and inflammation of the perianal area. The HemZem pessary pads or truss contain micropores which absorb and subsequently dry out the intestinal and hemorrhoidal mucus over time. As the mucus dries out, this natural in-place process creates a low-tack adhesive bond between the HemZem invention and the surrounding anal tissue. This, in addition to the anal and pelvic muscles and structural anatomy, holds the HemZem securely in place. The low-tack nature of the adhesive bond enables the invention to remain in place and then be removed without difficulty with fingers at an appropriate time following usage.

Because of these unique features the invention is novel, efficient, convenient to wear, unobtrusive, and not apparent to others when worn. In addition, because it is so small and light a user of the invention is in complete comfort and tends to forget that the invention is seated in place. The invention is also hygienic meaning that it can be disposed of by means of regular household plumbing. This has an advantage that a HemZem can be changed and gotten rid of in a regular toilet either at home or when a user is out and about in the community.

A comprehensive search of devices patented in regard to hemorrhoids by the US Patent Office shows that none either alone or in combination has the unique set of characteristics contained in the present HemZem invention.

Directions for use: HemZem Anal minipads are one-time use items. To be used once and then discarded. They contain no medication. They work by cleverly using simple body physics and chemistry to hold hemorrhoids in place enabling hemorrhoids to retract and shrink naturally. Properly inserted HemZem Anal minipads almost never fall out; they are kept in place by the continuous contraction of the anal sphincter muscle, the muscles of the pelvic floor, and the adhesive nature of bodily secretions. This permits regular daily activities without worry.

Following defecation, carefully clean anal area. Stand upright and gently compress any prolapsed piles back into anus by means of toilet or other soft tissue and flat fingers of wiping hand. (HemZem is not recommended or approved for this purpose.) Bend knees and hips slightly keeping feet flat on floor to facilitate separating the buttocks and reaching the anus.

Remove one HemZem from packet and keep folded. Take and hold HemZem between thumb and middle finger shaped as an inverted “V.” Reach behind to position it directly on the anus. Apply pressure with extended middle or index finger (or other finger if desired) to slide HemZem about a half inch into anus so that it sits firmly and the remainder of HemZem protrudes outside. A smooth rocking motion facilitates insertion to lock HemZem and hemorrhoids into place. If there is excess resistance, withdraw HemZem and start over. Take some deep slow breaths to relax anal sphincter before restarting.

HemZem may be left in place for up to 8 hours or until it begins to disintegrate due to moisture and mucus absorption. Then pull it out with fingers and replace with fresh pad. Prior to defecation, remove HemZem and replace afterward with fresh unit. HemZem may be inserted using rubber finger stalls or protectors. Used HemZem is flushable like toilet tissue. Be sure to wash hands following HemZem insertion.

Flatus (gas) may be expelled without dislodging the HemZem if the flatus is gently eased out. Avoid forceful expulsion.

Caution: If infection is present, HemZem should not be used without first consulting a licensed medical provider. Prolapsed hemorrhoids that cannot be pushed back through the anus require medical care, as does continued bleeding. HemZem should not be left in place for longer than 12 hours at a time.

There are five (5) claims for the HemZem Anal Pile Driver Medical Device. The inventor of the device claims that the device:

1. keeps hemorrhoids in their normal anatomical position and prevents recurrent hemorrhoid prolapse.

2. Absorbs mucus associated with prolapsed hemorrhoids.

3. Eliminates medical complications associated with prolapsing hemorrhoids, viz perianal irritation, itching, burning, pain, and discomfort.

4. Prevents soiling of underwear from mucus, and anal effluvia,

5. Prevents soiling of underwear following the use of topical anal or hemorrhoid creams, gels, and ointments.
